(19) United States
(12) Patent Application Publication
(10) Pub. No.: US 2004/0142341 A1
(43) Pub. Date: Jul. 22, 2004

(54) System for measuring membrane permeation

(76) Inventors: Johannes Schmitt, Leipzig (DE); Joachim Noller, Leipzig (DE)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/475,888

(22) PCT Filed: Apr. 24, 2002
(86) PCT No.: PCT/EP02/04492

(30) Foreign Application Priority Data
Apr. 27, 2001 (DE) 101 21 903.2

(51) Int. Cl. C12Q 1/68; C08G 63/48; C08G 63/91; B32B 5/16

(52) U.S. Cl. 435/6; 525/54.2; 428/403

(57) ABSTRACT

The invention relates to a system, which can be used for measuring membrane permeation in substances. Said system comprises, essentially, porous particles with an inner surface formed within the pores, and another outer surface. Essentially, only the outer surface is fully covered by a lipid layer, said lipid layer extending over the openings of the pores on the outer surface. Preferably, an intermediate layer is arranged between the outer surface and the lipid layer. Said intermediate layer is embodied, more particularly, in the form a polymer network.

The invention relates to porous particles which are surrounded by a lipid layer and to the use of these particles for measuring the membrane permeation of substances.

In many fields of research, it is necessary to characterize the ability of different substances to traverse membranes. In a general manner, behavior in relation to membranes and/or lipids is an important aspect when investigating biomolecules, in particular peptides or proteins. To a very particular extent, the ability of substances to traverse membranes plays an important role in pharmaceutical research in connection with finding and characterizing active compounds. A quite crucial point for being able to use an active compound in the field of medicine is the extent to which this active compound is able to penetrate membranes and in this way, for example, reach the interior of cells. Model systems which are suitable for this field, i.e., what is termed pharmacokinetics, have been sought and investigated for a long time now. The intention is for these models to make it possible to imitate the natural conditions in the organism, as regards the membranes which are present therein, to the extent that these models can then be used to make reliable assertions as regards the ability of the given substances to traverse membranes in vivo.

The permeation of substances through membranes or through lipid layers is essentially based on passive and active transport mechanisms. A variety of elements within the membrane or the lipid layer are of crucial importance for active transport. These elements are, in particular, transport proteins without which a variety of substances would not be able to pass through a membrane at all. Ion channels, which are present in membranes, which are also essentially formed from proteins and which permit and control the passage of ions, are also important in this connection.

The methods which have thus far been established for experimentally determining the permeation of substances through membranes, in particular through biomembranes, can be subdivided, in regard to the membrane morphology which is used for this purpose, into planar and spherically curved membrane systems.

The planar membrane systems are in the main arranged at the contact site between two aqueous compartments A and B, which are otherwise completely separate, and make it possible to measure the passage of substances from compartment A to compartment B using conventional methods. The membrane systems include the black lipids membranes (BLMs) (Wardak, A., Brodowski, R., Krupa, Z., Gruszecki, W. I., (2000) Journal of Photochemistry and Photobiology B 56, 12-18), membranes in filter pores (Kansy, M., Sermer, F., Gubernator, K. (1998) Journal of Medicinal Chemistry 41, 1007-1010 and Schmidt, C., Mayer, M., Vogel, H., (2000) Angewandte Chemie Int. Edition 39, 3137-3140) and the class of solid body-supported membranes (Cornell, B. A., Braach-Maksvyits, V. L., King, L. G., Osman, P. D., Raguse, B., Wiecorek, L., Pace, R. J., (1997) Nature 387, 580-583). Due to their morphology, membrane systems of this nature can be used for biosensor applications. However, in the case of the solid body-supported membranes, the size of compartment B is generally very small as compared with that of compartment A, resulting in it being possible for the permeation of substances through the membrane to be affected as a consequence of the uptake capacity of compartment B being limited.

Another crucial disadvantage of these planar membrane systems is that the membrane surface which is available for the substance exchange between the two compartments A and B is small overall. This applies particularly to what are termed the patch-clamp techniques, in which microscopically small regions of natural or artificial membranes are stretched over a pipette tip and transport of the substance or ion is detected electrically (Bordi, F., Carnetti, C., Motta, A., (2000) Journal of Physical Chemistry B, 104, 5318-5323).

Because of these disadvantages, it has thus far in the main only been possible to employ planar membrane systems usefully for detecting the permeation of ions through membranes. It has only been possible to use planar membranes in filter pores for measuring the permeation of other substances (Kansy, M., Sermer, F., Gubernator, K. (1998) Journal of Medicinal Chemistry 41, 1007-1010). However, a general problem of these planar systems is always the undefined nature of the given membranes or lipid layers. In this connection, it is not possible to verify whether one is dealing, for example, with a lipid double layer or with what are termed multilayers. Since permeation measurements of this nature are carried out in order to obtain information about the behavior of substances under natural conditions, it is essential that defined lipid layers, that is, in particular, lipid double layers, are used. If this cannot be guaranteed, it is not possible, in the first place, to achieve any reproducible results and, in the second place, the results which are obtained, have little informative value as regards making predictions about the behavior of the substances under natural conditions.

The spherically curved membrane systems include what are termed the liposomes or vesicles, which separate an outer compartment A from an internal compartment B. This compartment B is located within the liposomes or vesicles and is consequently also located within compartment A. Because of their colloidal dimensions, these systems have the advantage, as compared with the planar systems, of a substantially larger membrane interface between compartments A and B, thereby making it possible, in particular, to measure substances which permeate slowly. In addition, this can thereby drastically lower the error rate of individual membrane measurements. A variety of detection methods have thus far been reported as being used for permeation measurements which employ these systems. These methods include, for example, fluorescence (Sigler, A., Schubert, P., Hillen, W., Niederweis, M., (2000) European Journal of Biochemistry 267, 527-534), radioactivity and electrical measurement methods (Hill, W. G., Zeidel, M. L., (2000) Journal of Biological Chemistry 275, 30176-30185). These detection methods can be used for detecting in a time-resolved manner the permeation which is taking place. Permeation measurements carried out on individual liposomes have also been described (Olbrich, K., Rawicz, W., Needham, D., Evans, E., (2000) Biophysical Journal 79, 321-327). However, measurements of this nature are technically very elaborate and susceptible to error and therefore not suitable for routine measurements.

The general disadvantage of the conventional spherically curved membrane systems is their instability, which makes reliable and reproducible measurements, particularly within the context of serial investigations, virtually impossible. Furthermore, the known spherically curved membrane systems cannot be defined morphologically with regard to the size and number of the lipid layers. As regards their morphology, the liposomes and vesicles which are used in this connection are, if anything, a random product, such that reliable and reproducible measurements are generally not possible.

In order to circumvent the problem of instability, it has been proposed that hollow spheres which are coated with lipid membranes and which are prepared from a stable mesh should be used for measuring permeation (Moya, S., Donath, E., Sukhorukov, G. B., Auch, M., Baumler, H., Lichtenfeld, H., Möhwald, H., (2000) Macromolecules 33, 4538-4544). This makes it possible to provide relatively stable membrane systems. However, these coated hollow spheres are not suitable for measuring the membrane permeation of substances in an automated manner. On the one hand, it is not possible, in this present case, to equip compartment B, that is the interior of the hollow spheres, with different functionalities which would make it easier to detect the permeating substances. On the other hand, the density of the coated hollow spheres is so low that it is only possible with relatively great effort to isolate the spheres from an aqueous phase, for example. Isolating the membrane, system in this way would be a prerequisite for rapidly and reliably analyzing the permeated substances.

The invention therefore sets itself the object of providing a model system for membranes, in particular for native membranes, which can be used to analyze the permeation of substances through membranes or lipid layers. In this connection, the membranes or the lipid layers should be defined sufficiently precisely to enable reliable and reproducible results to be achieved. Furthermore, the system should be stable. In addition, the system should possess properties which are such that it is suitable for automated processes. Finally, the invention sets itself the object of creating a membrane system which is sufficiently flexible as to enable it to be adapted to a very wide variety of experimental conditions, in particular detection methods.

This object is achieved by means of porous particles as described in claim 1. Preferred embodiments of these particles are explained in claims 2-16. Claims 17-29 relate to a process for preparing the novel particles. Claims 30-32 deal with a process for using these particles to measure the permeation of substances. Claims 33-35 relate to the use of the particles or of a kit for measuring the membrane permeation of substances or for investigating membrane components. The wording of all the claims is hereby incorporated into the description by reference.

The novel porous particles possess an internal surface, which is formed within their pores, and an external surface, which is formed by the remainder of the surface. These particles are characterized by the fact that they are completely covered by a lipid layer, with this lipid layer essentially covering the external surface of the particles and thereby spanning the openings of the pores at the external surface. Consequently, the lipid layer essentially does not penetrate into the pores. This thereby creates a system which separates, by means of the lipid layer, a compartment A outside the particles from a compartment B within the pores. As a consequence of its particulate structure, the system is a dispersible 2-compartment system. This system is particularly suitable for measuring membrane permeation. In order to investigate the membrane permeation of substances, the system is brought into contact with liquids and the substances which are dissolved therein. The substances which are dissolved in the liquids penetrate the lipid layer, in dependence on their membrane permeation properties, and in this way come to be located in the pore volume of the particles. After the permeated substances have entered the pore volume, they can be analyzed quantitatively, thereby making it possible to determine the permeation constant of the substances. Advantageously, the lipid layer encloses the outer surface of the porous particles in an essentially impermeable manner. This is necessary so as to ensure that the substances to be analyzed are unable to penetrate into the pores by a route other than by way of the membrane.

The lipid layer which surrounds the particles is preferably a lipid double layer. The properties of a lipid double layer are very similar to those of native membranes, which means that this novel system can be used to recreate the natural conditions. In contrast to conventional systems, the morphology of the lipid layer can be controlled very precisely in the novel system. The system is therefore a precisely defined system, which constitutes the prerequisite for reliable and reproducible experimental results. From the outside, the novel particle system corresponds, in its morphology and its surface constitution, to liposomes or vesicles which are used conventionally for membrane permeation measurements. However, aside from other advantages, the novel system exhibits a substantially higher stability than do liposomes or vesicles and is therefore considerably better suited for membrane permeation measurements.

In a particularly preferred embodiment of the invention, an intermediate layer is provided between the surface, in particular the external surface, of the particles and the lipid layer. This intermediate ferably a network. The intermediate layer covers the particles without essentially penetrating into the pores. It primarily serves to form a support for the lipid layer so as to ensure that the lipid layer essentially only covers the external surface of the particles. The intermediate layer is constituted such that, as compared with the lipid layer, it does not significantly hinder the diffusive transport of solvent, in particular water, and the substances which are dissolved therein. In addition, the intermediate layer is preferably adsorbed or anchored relatively firmly on the surface of the particles in order to ensure that the novel particles are correspondingly stable over a long period. The intermediate layer is preferably constituted in such a way that it is able to take up water or another solvent. The thickness of the intermediate layer which can thereby be established creates a certain distance between the particle surface and the lipid layer. A distance of this nature is generally advantageous so as to ensure that the dynamic and structural properties of the lipid layer are not dominated by the proximity of the particle surface.

In a preferred embodiment of the invention, the intermediate layer consists at least partially of at least one polymer. Polymers composed of organic material are particularly preferred in this connection. In the present case, the term polymer also encompasses copolymers and block copolymers. Advantageously, the polymers are molecules having relatively long chains. This thereby ensures that, for steric reasons alone, the polymers span the openings of the pores in the external surface of the particles and essentially do not penetrate into the pores. Suitable polymers are polyelectrolytes, in particular anionic poly-electrolytes, polyampholytes, in particular proteins, DNA and/or RNA, and/or polyzwitterions.

In another preferred embodiment of the invention, the polymer is polystyrene sulfonate (PSS), in particular sodium polystyrene sulfonate, and/or poly(styrene-co-maleic anhydride) (PSPMA). These materials are also very suitable in accordance with the invention since, because of their long-chain structure, they essentially do not penetrate into the pores and surround the external surface of the particles with a network. Furthermore, these polymers take up water and/or other solvents to a certain extent and consequently ensure that there is a certain minimum distance between the particle surface and the lipid layer, thereby ensuring that the dynamic properties of the lipid layer are not impeded.

The intermediate layer can consist of a stack of different molecules, with the molecules preferably interacting with each other. The layer which lies closest to the particle surface is preferably fixed by means of adsorption and/or chemisorption.

The density or the aperture size of the intermediate layer is influenced, on the one hand, by the material which is selected for the intermediate layer. On the other hand, it depends on the conditions which are selected for preparing the intermediate layer, in particular the concentration of the material for the intermediate layer. The density of the aperture size of the intermediate layer is preferably selected such that free diffusion of the substances is not impaired and the carrier function of the intermediate layer is ensured. Consequently, it may be preferred for the aperture size to be relatively large. On the other hand, it can also be advantageous to select a narrower aperture size so as to ensure that the entire system is as a whole more stable. This thereby achieves higher pressure resistance, for example. This can be advantageous with regard to working with higher osmotic gradients and/or in connection with storage and/or transport properties.

In another preferred embodiment, the pores of the particles contain compounds and/or are, in particular, essentially filled with the compounds. Compounds which are suitable for this purpose do not bring about any significant restriction of the diffusive transport of substances within the pores. The compounds within the pores fulfill a certain supporting function for the intermediate layer and/or the lipid layer. The material of the intermediate layer, in particular the polymers, does not, in this case, have to span the pores without any support. Materials having relatively short chains, in particular short-chain polymers, can therefore also be suitable for preparing the intermediate layer. In a particularly preferred embodiment of the invention, the intermediate layer can be dispensed with because of the supporting function of the compounds within the pores, which means that the lipid layer is immobilized directly on the external surface of the particles with the pore openings at the external surface in this case also being spanned by the lipid layer. The embodiment using compounds within the pores has the crucial advantage that the pressure resistance of the system can be markedly increased.

The compounds within the pores are preferably polymers, in particular polymers composed of organic material. In this connection, the polymers also include copolymers and block copolymers. Particular preference is given to polyelectrolytes, polyampholytes, in particular proteins, DNA and/or RNA, and/or polyzwitterions. Fluorescence probes and/or luminescence probes, which can be used for detecting the permeated substances when carrying out the permeation measurement, are very particularly suitable. In a preferred embodiment of the invention, the compounds within the pores are not water-soluble and can consequently serve as a matrix for introducing other hydrophobic molecules into the pores. The compounds can be fixed by means of adsorption and/or chemisorption. The compounds can furthermore enable other molecules to be chemically bonded, adsorbed or enclosed. The intermediate layer, or the filling in the pores, preferably exhibits other molecules, in particular functional molecules.

In a preferred embodiment of the invention, the surface, in particular the internal surface, of the particles is modified. In this way, it is possible, for example, to provide a surface which, taken overall, is hydrophobic (passive) and which is suitable for applying other molecules, in particular molecules possessing hydrophobic functionalities, by means of adsorption and/or chemical bonding. A hydrophobic internal surface can be achieved, for example, by applying a silane layer. Aside from such a passivation, an activation may also, for example, be preferred, resulting in the surface being prepared in such a way as to enable what is essentially a selective chemical reaction with other molecules, for example with proteins, to take place. In such an embodiment, the surface can, for example, be modified with cyanogen bromide. In other preferred embodiments, the internal surface is modified with amino, epoxy, halogenyl and/or thio groups. Mercaptans and/or disulfides, in particular alkyl disulfides, are, for example, used for the modification. Particularly preferred examples are N-(2-aminoethyl)-3-aminopropyltrimethoxysilane (EDA), polyethyleneimine (PEI) and/or cysteamine, in particular cysteamine hydrochloride.

In a preferred embodiment of the invention, the surface, in particular the internal surface, exhibits functional molecules. These functional molecules can interact directly with the surface of the particles and be fixed in this way. However, the functional molecules are preferably fixed by way of an interaction with a modified surface. This naturally depends, inter alia, on the given material or the surface of the particles and on the functional molecules which are to be applied. Furthermore, the functional molecules can be fixed due to interactions with the intermediate layer or with the filling in the pores.

Using hydrophobic functionalities within the particles makes it possible, according to the invention, to construct a system which possesses a hydrophilic compartment A and a hydrophobic compartment B at whose interface there is a lipid layer which controls transport between the different compartments.

The functional molecules are preferably molecules which are connected with detecting the permeated substances during the permeation measurement. The functional molecules are preferably molecules which are enzymically, optically and/or chemically, in particular photochemically, active. Particular preference is given, in this connection, to molecules which are suitable for detecting the permeated substances by means of fluorescence and/or luminescence. Very particular preference is given, in this connection, to the resonant energy transfer detection method, in which a fluorescence donor molecule and a fluorescence acceptor molecule enter into interaction with each other. For this purpose, either the donor molecule or the acceptor molecule is fixed within the particles. The substances which are to be analyzed now constitute the corresponding fluorescence partner, that is the acceptor molecule or the donor molecule. After the substances have entered into the pores through the lipid layer they then enter into interaction with what is at that time the other partner molecule and in this way give rise to a fluorescence signal which can be analyzed.

It is not a prerequisite for the invention that only the internal surface of the particles is modified and/or functionalized. If the external surface of the articles were also to be modified or functionalized it is in any case ensured that the substances which were to be analyzed during the permeation measurement would first of all have to traverse the lipid layer before they interacted with these functionalities.

The lipid layer which surrounds the porous particles can be varied to a very great extent. In principle, any compositions of the layer are possible, with the layer essentially consisting of amphiphilic molecules. Particular preference is given to a lipid layer which is at least partially composed of lipids, lipid derivatives, lipid-analogous substances and/or native membranes, in particular plasma membranes. Using native membranes, or fragments of such membranes, as a constituent of the lipid layer has the advantage, in the first place, that this reflects the natural situation. In the second place, this natural situation does not need to be analyzed in detail, particularly with regard to the different constituents of the membranes.

This diversity of the lipid layer represents a crucial advantage of the invention since the predetermined particle geometry ensures that each lipid layer composition which is applied to the particles exposes the same shape and size to compartment A. This is not the case, for example, with regard to the use, which is known from the prior art, of liposomes or vesicles since their morphology crucially depends on the composition of the given lipid layer.

Particular preference is given to the lipid layer exhibiting other substances, in particular peptides, proteins, nucleic acids, surfactants and/or polymers. Such compositions of the lipid layer make it possible to reflect the natural conditions of a membrane virtually identically. As a result, the membrane system according to the invention provides an optimal model system for natural membranes.

Preference is furthermore given, according to the invention, to the lipid layer exhibiting transport elements. These include, in particular, transport proteins, for example peptide transporters, pore-formers and/or ion channels. Pore formers are to be understood as being substances which generate holes in membranes. The entire thickness of the lipid layer can preferably be spanned by special molecules which are able to perform a transport function for substances which are dissolved in liquid phases. This makes it possible, on the one hand, to imitate the natural conditions of a membrane. On the other hand, it makes it possible to specifically analyze the interaction of particular substances with particular transport elements. Thus, it is possible, for example, to investigate the conditions under which a transport protein or an ion channel displays its optimal activity.

In a particularly preferred embodiment of the invention, the porous particles are porous spheres. Advantageously, the spheres have a diameter of from about 1 to about 100 μm, in particular from about 3 to about 10 μm.

The particles, in particular the spheres, preferably possess pores having an opening width of from about 1 to about 1000 nm, in particular from about 5 to about 50 nm. Suitable nanoporous particles preferably possess a defined pore size such that using particles of a particular pore size makes it possible to selectively control the properties of the novel system. Depending, in particular, on the detection methods which are chosen in each case, it may be preferable to use larger or smaller pore sizes or other particle diameters. For example, using a relatively small pore diameter can increase the sensitivity of local probe molecules, e.g. dyes, which are incorporated. Thus, in the case of the resonant energy transfer detection method which has already been mentioned, typical interaction distances between donor and acceptor molecules in the range of approx. 5 nm are virtually optimal. When the pore diameter of the novel particles is 10 nm, for example, each permeated molecule (e.g. donor molecule) then inevitably interacts, after having passed through the lipid layer, with the molecules (e.g. acceptor molecules) which are immobilized on the pore wall. By varying the pore diameter it is possible, in this way, to “fine-tune” the interaction.

In a preferred embodiment of the invention, the internal surface of the pores is equipped with fluorescence probes whose fluorescence reacts sensitively to the proximity of a particular substance, especially a particular molecule or ion, which has permeated from compartment A to compartment B. The large internal surface of the porous particles makes it possible, in this way, to achieve a high fluorescence yield which conventional fluorescence-spectroscopic methods can exploit for sensitively detecting the permeation process in a time-resolved manner.

In addition to this, using the novel porous particles for measuring membrane permeation has the additional advantage that, because of the small size of the pores, diffusion in the pores essentially takes place two-dimensionally and consequently more rapidly than in the conventional three-dimensional systems. By suitably selecting the pore diameter, it is also possible to adjust the average distance of the permeated substances from the probe molecules, thereby guaranteeing optimal interaction and efficient detection.

In one embodiment of the invention, the porous particles consist at least partially of inorganic material, in particular of silicon oxides, aluminum oxides and/or titanium oxides. In another preferred embodiment, the porous particles consist at least partially of organic material, preferably latex.

In a particularly preferred embodiment, the porous particles consist at least partially of silicate. The SiOH groups which are located on the surface of porous silicate particles can advantageously be used for functionalizing the surface with suitable molecules. Covalent bonds between surface groups and molecules are particularly preferred in this connection. In addition, molecules having a positive excess charge (e.g. polycations) can be adsorbed firmly on the surface by means of electrostatic interaction. The porosity of the particles, which is extremely high in the case of silicate particles, enables the internal compartment (compartment B) to have an internal surface which is enormous as compared with the external dimensions and which is available for fixing functional groups.

Porous silicate is a mechanically rigid material having a negative surface charge. Microscopic particles, in particular spheres, composed of silicate are therefore outstandingly dispersible in solution, particularly in aqueous solution. At the same time, due to their density, they are able to sediment under normal gravimetric conditions. This means that it is preferably possible to dispense with centrifugation when carrying out membrane permeation measurements. Nevertheless, a centrifugation may be advantageous under certain conditions in order, for example, to shorten the course of sedimentation.

In another preferred embodiment of the invention, the porous particles possess a magnetic core. Particular preference is given, in A this connection, to porous silicate spheres which have a magnetic core. This can thereby accelerate the sedimentation of particles, for example with regard to automating the permeation measurement process.

In the porous silicate particles which are preferred in accordance with the invention, the arrangement of the pores within the particles is relatively random. There is consequently no unambiguous definition of whether the pores communicate with each other completely or not. However, it may be advantageous to use particles which possess pores which are defined so as to guarantee that the pores form a communicating system. Equilibria can then advantageously be reached more rapidly within the particles and particular reactions can in this way be optimized within the particles.

The invention furthermore encompasses a process for producing porous particles having an internal surface which is formed within the pores and a remaining external surface, with essentially only the external surface being completely covered by a lipid layer, in particular a lipid double layer, and the lipid layer spanning the openings of the pores at the external surface. This process is characterized in that the pores of the particles are spiked, in particular essentially filled, with compounds and/or the porous particles are provided with a layer, in particular with a network. In a further process step, the particles which have been treated in this way are provided with a lipid layer, in particular with a lipid double layer. The reader is referred to the above description in regard to various details of the novel process.

Advantageously, the surface, in particular the internal surface, of the particles is modified, in particular passivated or activated, as described above

Furthermore, the surface can be provided with functional molecules, for example probe molecules (functionalization). The functionalization can “refunctionalize” the groups which have been introduced by the modification. The functional molecules can be fixed, for example, by means of chemisorption or adsorption. In a preferred embodiment, the surfaces are initially modified and/or functionalized and then provided with the layer, that is the intermediate layer. This intermediate layer preferably constitutes a network which consists, in particular at least partially, of polymers.

According to the invention, it is particularly preferred to perform the modification or functionalization of the surface after a coating of the particles has taken place. A prerequisite for this procedure is that the density or aperture width of the intermediate layer should be sufficiently large to enable the modifying or functionalizing compounds to pass through. This approach is particularly preferred when using probe molecules which are envisaged for a fluorescence detection. In general, it depends on the materials which are in each case selected, in particular the material of the intermediate layer and the material for the modification or functionalization, as to whether this procedural sequence is advantageous. As far as the technical aspects of producing particles are concerned, subsequently functionalizing or modifying the surface has very great advantages since, in this way, it is possible to simplify the entire process for producing the novel particles. For example, all the compounds which are required for producing the novel particles prior to applying the lipid layer can be added to the particles in one mixture.

After the layer, that is the intermediate layer, has been applied and/or after the pores of the particles have been spiked or essentially filled with compounds, a lipid layer is applied. The lipid layer is advantageously applied after any modifications and/or functionalizations of the particle surfaces have been undertaken. In order to produce the lipid layer, vesicles are prepared from lipids, lipid derivatives, lipid-analogous substances or native membranes, in particular plasma membranes. When the vesicles are being prepared, it is also possible for other substances, such as peptides or proteins, and also transport elements, to be present and thereby incorporated into the vesicles. The vesicles are prepared, and the lipid layer is applied to the particles, using conventional methods as described, for example, by Schmitt, J., Danner, B., Bayerl, T. M., (2000) Langmuir 17, 244-246.

The invention furthermore encompasses a process for using the novel porous particles to measure the membrane permeation of substances. To do this, the substances to be investigated are brought into contact with the novel porous particles in one mixture. After a certain incubation period, which is advantageously precisely defined, the quantity of the substances which have penetrated through the membrane is analyzed. In this way, it is possible to draw conclusions with regard to the membrane permeation property of the given substance.

The quantity of the substances which have passed through the membrane into the interior of the particles, in particular into the pores, is determined directly and/or indirectly. In a particularly preferred embodiment of this process, the particles are, after the incubation period, separated off from the remaining mixture and the quantity of the substances which is present within the particles is then determined. In another embodiment, the quantity of the substances which is present in the remaining mixture, after the particles have been separated off from the mixture, is determined. The two procedures can advantageously be combined with each other. The particles can be separated off in a variety of ways, for example by means of centrifugation and/or filtration. Particular preference is given to a “natural” sedimentation since this considerably simplifies the process of permeation measurement. However, it can be advantageous to accelerate the process, in which case centrifugation can be advantageous.

In a very particularly preferred embodiment of the invention, magnetic particles are employed as described above. As a result of this magnetic property, the particles can be separated off very rapidly and efficiently. This is advantageous particularly with regard to automating the entire process. When magnetic particles are used, the particles are separated off with the aid of a suitable magnet and the remaining mixture and the particles are then, separately from each other, available for further analysis. The centrifugation step, which is time-consuming and not readily accessible to automation, is thereby dispensed with. When the permeation measurement is automated, the process can, for example, be carried out in microtiter plates, with a large number of automation aids already being available for such microtiter plates. In this case, the incubation and detection of the permeation process preferably take place in the same vessel.

In a preferred embodiment of the novel process, the substances to be analyzed are determined by means of chemical, electrical, magnetic, radioactive or optical, in particular fluorimetric or luminometric, detection methods. For example, the membrane permeation of fluorescence-labeled substances can be investigated by adding the substances, in dissolved form, to compartment A, that is to the dispersed particles. After defined incubation periods, the particles are separated off from the remaining mixture and the fluorescence intensity of identical quantities of spheres is determined, preferably as a function of the incubation period, using conventional measurement methods. In another preferred embodiment of the novel process, use is made of fluorescence probes which are fixed in compartment B. Their fluorescence is sensitive to particular substances, especially molecules or ions, which are dissolved in compartment A. Permeation of these molecules or ions from compartment A to compartment B brings about a change in the entire fluorescence intensity of the dispersion. This can be measured as a function of the incubation period. In this embodiment, it is not necessary to separate off the particles from the remaining mixture after the incubation period.

In another embodiment, radioactively labeled substances are used in compartment A. After incubation has taken place, the detection is carried out by using suitable radioactive measurement methods to determine the radiation intensity which is being emitted by the spheres. This implementation example has the advantage that the detection options are extremely sensitive and that the substance to be investigated is only altered slightly by the incorporation of the given radioactivity.

In another embodiment of the invention, enzymes are immobilized in compartment B in such a way that they preferably retain their activity. Permeating substances which arrive in compartment B from compartment A and consequently arrive in the vicinity of the enzymes, transfer the enzymes to a different state which can be detected optically, for example by means of fluorescence or luminescence, or else electrically.

The novel process can be carried out in such a way that the permeated substances are analyzed after a certain incubation period. However, particular preference is given to determining the permeation as a function of the incubation period, i.e. to analyzing the permeated substances either as an end point determination after different, defined incubation periods or else to using suitable detection methods to monitor the permeation in a continuous assay.

In another embodiment, use is made of particles which, in compartment B, possess enzymes for which the substances to be analyzed are substrates. After the substances have permeated, the enzyme then converts the substances into products. Either the release of the products in compartment B leads itself to a change which can be detected optically or electrically, or the enzyme is induced to effect such a change and/or the products can be detected using other methods.

In a further embodiment, unlabeled substances, whose permeation is to be investigated, are used in compartment A. In this case, a detection [lacuna] using methods such as HPLC/UV vis spectroscopy or HPLC/mass spectroscopy to determine the quantity of the substance remaining in compartment A after an incubation period and after the particles have been separated off. A crucial advantage of this implementation example is that the substance to be investigated does not have to be labeled and/or specially purified and can nevertheless be detected with high sensitivity.

The invention furthermore encompasses the use of porous particles, as described above, for measuring membrane permeation.

The invention furthermore encompasses a kit for measuring the membrane permeation of substances. This kit contains components which are suitable for producing porous particles according to the invention as described above. In this case, it is not necessary for the kit to contain all the components for producing the novel particles. On the contrary, it can be preferable for only some, in particular essential, components to be present in the kit and for the other components to be provided by the user himself. In addition, the invention encompasses a kit for measuring the membrane permeation of substances with the kit preferably containing completely coated porous particles in conformity with the above description. This is particularly preferred when the particles are provided with a relatively complicated lipid layer that is, in particular, a lipid layer which, in addition to lipids, also contains other substances, such as proteins, in particular transport proteins, for example.

Finally, the invention encompasses a kit for investigating membrane elements, in particular proteins. The novel kit can advantageously be used to carry out functional investigations. For example, such a kit can be used for characterizing transport proteins.

The invention has crucial advantages when compared with previously known systems or processes for measuring membrane permeation. This applies, in particular, as regards stability, mechanical rigidity, morphology, detection and ability to separate from a dispersion. It is very advantageously possible to automate the permeation measurement. The novel particles are distinguished by novel options for functionalizing the surfaces and consequently options which can be used for detection.

The features of the invention which have been described, and additional features, ensue from the following examples, figures and subclaims. In this connection, each of the different features can either be realized on its own or in combination with other features.

The Figures Show:

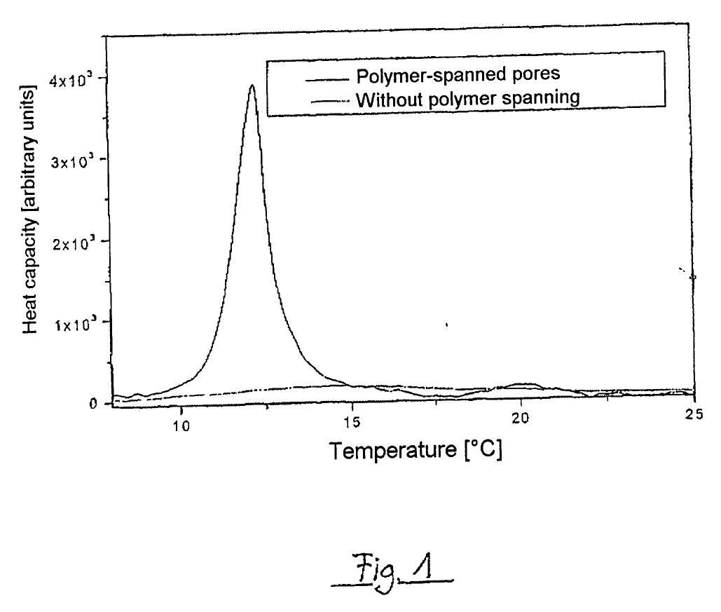

FIG. 1: differential calorimetric plots of dielaidoyl phosphatidylcholine (DEPC)-coated porous silicate particles with and without spanned pores,

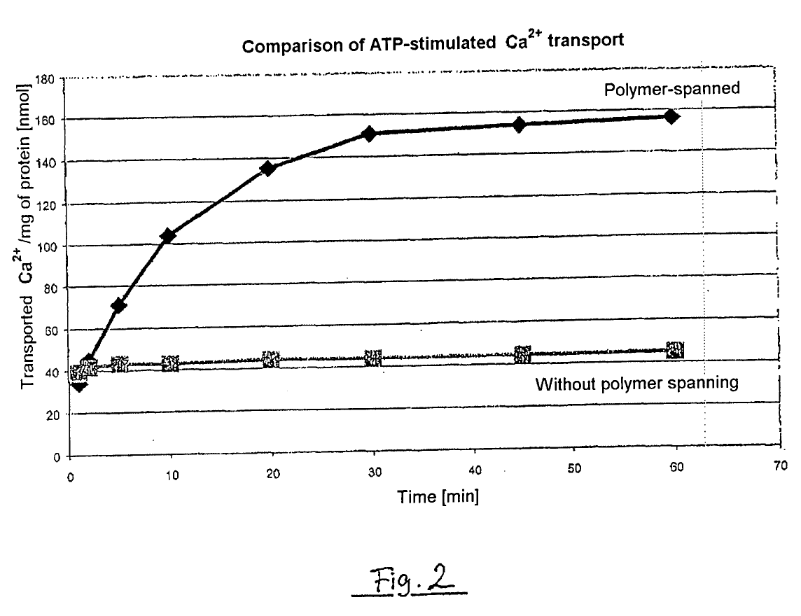

FIG. 2: measurements of ATP-stimulated Ca²⁺ transport performed on immobilized sarcoplasmic reticulum Ca²⁺-ATPase in the case of lipid-coated silicate particles with and without an intermediate polymer layer.

### EXAMPLES

### Example 1

### Functionalizing the Solid Body Surface

1.1. Amino-Functionalizing Pulverulent and Porous Silicate Surfaces with N-(2-aminoethyl)-3-aminopropyltrimethoxysilane (EDA)

A silane solution, consisting of 9.2 ml of N-(2-aminoethyl)-3-aminopropyltrimethoxysilane (EDA) and 243 μL of concentrated acetic acid in 450 ml of deionized water, is prepared fresh. After 5 minutes, 2 g of a porous silicate material (Nucleosil 50-10 from Macherey-Nagel, Düren) are added to the silane solution and the material is suspended by shaking. This dispersion is slowly rotated for three hours; after that, the silicate material is sedimented and washed three times with deionized water. The success of the silanization is documented by means of diffuse-reflectance infrared spectroscopy (DRIFT), which is performed on the dried silicate material. Other support materials having pore sizes of between 5 and 400 nm are functionalized in a similar manner.

1.2. Amino-Functionalizing Pulverulent and Porous Silicate Surfaces with Poly(diallyldimethyl-ammonium Chloride)

1 g of a porous silicate material (Nucleosil 50-3 from Macherey-Nagel, Düren) is added to a poly(diallyldimethylammonium chloride) (polyDADMAC, Mw about 400,000-500,000) solution consisting of 200 ml of polyethyleneimine (PEI) (20% solution in water, Aldrich, Steinheim) in 50 ml of a 3M solution of NaCl, and the mixture is slowly rotated for three hours. After that, the silicate material is sedimented and washed three times with deionized water. The success of the reaction is documented by means of diffuse-reflectance infrared spectroscopy (DRIFT), which is performed on the dried silicate material.

### Example 2

### Incorporating Functional Molecules Prior to Spanning

2.1. Immobilizing an Enzyme (Esterase)

1 g of an EDA carrier material which has been functionalized as described in Example 1.1. is added to an esterase solution consisting of 30 mg of ESTERASE (E.C. 3.1.1.1., activity 20 units/mg) in 15 ml of phosphate buffer (20 mM, pH 7.4), and the mixture is rotated overnight. After that, the carrier material is sedimented and washed three times with phosphate buffer. The success of the treatment is documented by the decrease in the esterase in the solution and by means of carrying out measurements of the activity of the enzyme which is immobilized on the carrier material.

2.2. Introducing a Probe Molecule

50 mg of an EDA carrier material which has been functionalized as described in Example 1.1. are added to 1 ml of a 1 mM solution of Quin2 (Calbiochem, Bad Soden) in TEA buffer (50 mM TEA, 25 mM NaCl), and the mixture is rotated for one hour. After that, the carrier material is sedimented and washed three times with TEA buffer. The success of the treatment is documented by the decrease in Quin2 in the solution and by means of carrying out fluorescence measurements.

### Example 3

### Spanning the Porous Spheres with Polymers

3.1 Adsorbing Na Poly(Styrenesulfonate) (PSS) on EDA-Functionalized Silicate Surfaces

1 g of a silicate material which has been amino-functionalized as described in Example 1.1. is added to a Na poly(styrenesulfonate) (PSS) solution consisting of 25 mg of PSS (Mw approximately 2,600,000, FLUKA) in 50 ml of deionized water and the mixture is shaken for three hours. After that, the silicate material is sedimented and washed three times with deionized water. The success of the adsorption is documented by means of DRIFT and from the decrease in the concentration of PSS in the solution.

3.2. Adsorbing Na Poly(Styrenesulfonate) (PSS) on Poly(Diallyldimethylammonium Chloride)-Functionalized Silicate Surfaces

1 g of a silicate material which has been amino-functionalized as described in Example 1.2. is added to a Na poly(styrenesulfonate) (PSS) solution consisting of 25 mg of PSS (Mw approximately 70,000, Aldrich, Steinheim) in 50 ml of deionized water, and the mixture is shaken for three hours. After that, the silicate material is sedimented and washed three times with deionized water. The success of the adsorption is documented by means of DRIFT and from the decrease in the concentration of PSS in the solution.

3.3 Adsorbing Na Poly(Styrenesulfonate) (PSS) on Enzyme-Containing Silicate Surfaces

0.5 g of a silicate material which has been prepared as described in Example 2.1. is added to a Na poly(styrenesulfonate) (PSS) solution consisting of 12.5 mg of PSS (Mw approximately 2,600,000, FLUKA) in 25 ml of deionized water and the mixture is shaken for three hours. After that, the silicate material is sedimented and washed three times with deionized water. The success of the absorption is documented by means of DRIFT and from the decrease in the concentration of PSS in the solution. The integrity of the enzyme after the spanning is determined by performing activity measurements.

### Example 4

### Incorporating Functional Molecules After the Spanning

4.1. Preparing Photoreactive Surfaces on PSS/EDA-Functionalized Silicate Surfaces

0.25 g of an EDA/PSS carrier material which has been functionalized as described in Example 3.1. is added to a solution consisting of 0.25 g of 3,3′,4,4′-benzophenonetetracarboxylic dianhydride (BPA) in 25 ml of acetone, and the mixture is rotated overnight. After that, the carrier material is sedimented, washed three times with acetone and dried. The success of the treatment is documented by means of DRIFT.

4.2. Preparing Anhydride Surfaces on PSS/EDA-Functionalized Silicate Surfaces

0.25 g of an EDA/PSS carrier material which has been functionalized as described in Example 3.1. is added to a solution consisting of 0.1 g of 3,3′,4,4′-biphenyltetracarboxylic dianhydride in 25 ml of acetone, and the mixture is rotated overnight. After that, the carrier material is sedimented, washed three times with acetone and dried.

4.3. Introducing a Probe Molecule

50 mg of an EDA/PSS carrier material which has been functionalized as described in Example 3.1. is added to 1 ml of a 1 mM solution of Quin2 (Calbiochem, Bad Soden) in TEA buffer (50 mM TEA, 25 mM NaCl, pH 7.4), and the mixture is rotated for one hour. After that, the carrier material is sedimented and washed three times with TEA buffer. The success of the treatment is documented by the decrease in Quin2 in the solution and by means of performing fluorescence measurements.

### Example 5

### Depositing Lipid Layers on the Modified Surfaces

5.1. Preparing Lipid Vesicles for Immobilization on Pulverulent Surfaces

80 mg of dielaidoyl phosphatidylcholine (DEPC) are swollen, at room temperature for half an hour, in 16 ml of coating buffer consisting of 20 mM HEPES buffer, pH 7.1, containing 30 mM NaCl and then ultrasonicated for 30 minutes using a rod sonicator (Branson Sonorex). The result is a clear vesicle dispersion having vesicle diameters in the 20-80 nm range. The determination is effected by means of conventional dynamic laser light scattering (particle-sizing).

5.2. Immobilizing Lipid Membranes on Spanned Silicate Surfaces

1 g of a porous silicate carrier which has been spanned as described in Example 3.1. is added to 16 ml of a vesicle dispersion which has been prepared as described in Example 5.1, and the mixture is slowly rotated for 30 minutes. After that, the carrier material is sedimented and washed three times with coating buffer. The success of the coating is documented by means of DSC, which is carried out on the material which is dispersed in the coating buffer (as described in C. Naumann, T. Brumm, T. M. Bayerl, Biophys. J., 1992, 63, 1314), and DRIFT (after drying the material) and by determining the quantity of lipid.

5.3. Immobilizing Lipid Membranes on Enzyme-Containing Surfaces Exhibiting Mixed Functionality

1 g of a porous silicate carrier which has been spanned as described in Example 3.3. and which contains enzyme is added to 16 ml of a vesicle dispersion which has been prepared as described in Example 5.1, and the mixture is slowly rotated for 30 minutes. After that, the carrier material is sedimented and washed three times with coating buffer. The success of the coating is documented by means of DSC, which is carried out on the material which is dispersed in the coating buffer, and DRIFT (after drying the material).

5.4. Immobilizing Native Sarcoplasmic Reticulum (SR) Membranes on Spanned Silicate Surfaces and Measuring the Ca²⁺-ATPase Function

Membrane vesicles of the sarcoplasmic reticulum (SR vesicles) are prepared from the muscle tissue of a rabbit in accordance with a method of W. Hasselbach and M. Makinose (Biochem. Z. 1961, 333, 518-528). Ultrasonication is then used to convert this dispersion into small, single-shell vesicles having a diameter of 20-90 nm. 50 mg of a porous silicate carrier which has been spanned as described in Example 4.3. are added to 900 μl of this solution (about 0.5 mg of total protein), and the mixture is incubated at 4° C. for 18 hours using 100 mM triethanolamine (pH 7.4) and 100 mM NaCl as the buffer solution (incubation buffer). After that, the carrier material is sedimented and washed three times with incubation buffer. The success of the coating is documented by means of performing DRIFT on the dried material. The Ca²⁺-ATPase activity on the carrier material after the washing in the incubation buffer is [lacuna] by determining the ATP hydrolysis activity in dependence on the calcium ion concentration and the Ca²⁺ transport and its inhibition by the specific inhibitor cyclopiazonic acid. In order to measure the quantity of Ca ions which was transported, the fluorescence (excitation filter: 340 nm, emission filter: 510 nm) was measured continuously in a fluorescent plate reader (HTS7000, Perkin-Elmer) (FIG. 2). These function tests prove that the Ca²⁺-ATPase activity on the carrier material is comparable to that in an SR vesicle.

### Example 6

### Properties of the Membranes on Optimized Carrier Material

6.1. Stability in a Flowing Aqueous Medium

The systems described in Examples 5.2. to 5.4. are exposed, for a period of 24 hours, to a flowing medium (coating buffer as described in Example 5.1. or incubation buffer as described in Example 5.4.) in a test bath. In each case equal quantities of carrier material are removed from the test bath at intervals of 2 hours and dried, and DRIFT is then used to investigate their coating. In addition, DSC is used to investigate the systems described in Examples 5.2. and 5.3. A measurable decrease in the membrane coating with time is not observed either with DRIFT or with DSC.

6.2. Stability After Freezing

The systems described in Examples 5.2 to 5.4. are frozen at −80° C. in the dispersed state and then brought once again to room temperature and dried. Comparative DRIFT measurements which were performed before and after the freezing demonstrated that the quantities of lipid on the carrier material were unchanged.

6.3. Stability of the Enzymic Activity of SR-Coated Carrier Material

After having been prepared, the system described in Example 5.4. is stored at −80° C. for a period of 3 months. At intervals of 1 month, samples are removed and their Ca²⁺-ATPase activity is investigated using the method described in 5.4. After 2 months, the activity has declined down to approx. 70% of the original value (measured immediately after the carrier material was prepared and washed). It is not possible to measure any Ca²⁺-ATPase activity in the supernatant from the stored samples.

6.4. Determining the Phase Transition Temperatures

FIG. 1 shows differential-calorimetric (DSC) measurements of the phase transition of solid body-supported bilayers consisting of the synthetic lipid dielaidoyl-sn-3-glycero-3-phosphocholine (termed DEPC below) on an unspanned surface (preparation in accordance with C. Naumann, T. Brumm, T. M. Bayerl, Biophys. J., 1992, 63, 1314) and on a surface which as been spanned by means of the above step (as described in the example). These results show a marked broadening of the phase transition in the case of the unspanned surface. The phase transition temperature on the polymer-spanned surface corresponds to that of the DEPC vesicles.

1. A porous particle having an internal surface, which is formed within the pores, and a remaining external surface, with essentially only the external surface being completely covered by a lipid layer, in particular a lipid double layer, and the lipid layer spanning the openings of the pores at the external surface.

2. A particle as claimed in claim 1, characterized in that an intermediate layer, in particular a network, is provided between the surface, in particular the external surface, and the lipid layer.

3. A particle as claimed in claim 2, characterized in that the intermediate layer consists at least partially of at least one polymer, in particular of a polymer composed of organic material.

4. A particle as claimed in claim 3, characterized in that the polymer is a polyelectrolyte, in particular an anionic polyelectrolyte, a polyampholyte, in particular a protein, DNA and/or RNA, and/or a polyzwitterion.

5. A particle as claimed in claim 3 or claim 4, characterized in that the polymer is poly(styrenesulfonate) (PSS), in particular sodium poly(styrenesulfonate), and/or poly(styrene)-co-maleic anhydride (PSPMA).

6. A particle as claimed in one of the preceding claims, characterized in that the pores contain compounds, and are in particular essentially filled with compounds, with the compounds preferably being polymers, in particular polymers composed of organic material.

7. A particle as claimed in claim 6, characterized in that the polymers are polyelectrolytes, polyampholytes, in particular proteins, DNA and/or RNA, polyzwitterions, fluorescence probes and/or luminescence probes.

8. A particle as claimed in one of the preceding claims, characterized in that the surface, in particular the internal surface, exhibits modifications, in particular passivations or activations, with the modifications preferably being amino, epoxy, halogenyl and/or thio groups.

9. A particle as claimed in one of the preceding claims, characterized in that the surface, in particular the internal surface, exhibits functional molecules, with the functional molecules preferably being enzymically, optically and/or chemically, in particular photochemically, active molecules.

10. A particle as claimed in one of the preceding claims, characterized in that the lipid layer consists at least partially of lipids, lipid derivatives, lipid-analogous substances and/or native membranes, in particular plasma membranes.

11. A particle as claimed in one of the preceding claims, characterized in that the lipid layer contains other substances, in particular peptides, proteins, nucleic acids, surfactants and/or polymers.

12. A particle as claimed in one of the preceding claims, characterized in that the lipid layer contains transport elements, in particular transport proteins, pore formers and/or ion channels.

13. A particle as claimed in one of the preceding claims, characterized in that it is a porous sphere which preferably has a diameter of from about 1 to about 100 μm, in particular from about 3 to about 10 μm.

14. A particle as claimed in one of the preceding claims, characterized in that it possesses pores having an opening width of from about 1 to about 1000 nm, in particular of from about 3 to about 50 nm.

15. A particle as claimed in one of the preceding claims, characterized in that it consists at least partially of silicate and/or latex.

16. A particle as claimed in one of the preceding claims, characterized in that it possesses a magnetic core.

17. A process for producing porous particles having an internal surface, which is formed within the pores, and a remaining external surface, with essentially only the external surface being completely covered by a lipid layer, in particular a lipid double layer, and the lipid layer spanning the openings of the pores at the external surface, characterized in that
a) the pores of the particles are spiked, in particular essentially filled, with compounds and/or the porous particles are provided with a layer, in particular with a network, and
b) a lipid layer, in particular a lipid double layer, is applied to the particles which have been treated in accordance with process step a).

18. The process as claimed in claim 17, characterized in that polymers, in particular polymers composed of organic material, are at least partially used for spiking the pores with compounds and/or for preparing the layer.

19. The process as claimed in claim 18, characterized in that the polymers employed are polyelectrolytes, in particular anionic polyelectrolytes, polyampholytes, in particular proteins, DNA and/or RNA, polyzwitterions, fluorescence probes and/or luminescence probes.

20. The process as claimed in one of claims 17 to 19, characterized in that poly(styrenesulfonate) (PSS), in particular sodium poly(styrenesulfonate), and/or poly(styrene)-co-maleic anhydride (PSPMA) is/are at least partially used for preparing the layer.

21. The process as claimed in one of claims 17 to 20, characterized in that the surface, in particular the internal surface, is modified, in particular passivated or activated, before or after implementing process step a), with amino, epoxy, halogenyl and/or thio groups preferably being used for the modification.

22. The process as claimed in one of claims 17 to 21, characterized in that the surface, in particular the internal surface, is provided with functional molecules before or after implementing process step a), with the functional molecules employed preferably being enzymically, optically and/or chemically, in particular photochemically, active molecules.

23. The process as claimed in one of claims 17 to 22, characterized in that, for preparing the lipid layer in accordance with process step b), vesicles composed of lipids, lipid derivatives, lipid-analogous substances or native membranes, in particular plasma membranes, are prepared and brought into contact with the particles.

24. The process as claimed in one of claims 17 to 23, characterized in that the vesicles other substances, in particular peptides, proteins, nucleic acid, surfactants and/or polymers, are employed for preparing the lipid layer in accordance with process step b).

25. The process as claimed in one of claims 17 to 24, characterized in that transport elements, in particular transport proteins, pore formers and/or ion channels, are also employed for preparing the lipid layer in accordance with process step b).

26. The process as claimed in one of claims 17 to 25, characterized in that the particles employed are porous spheres, with the spheres preferably having a diameter of from about 1 to about 100 μm, in particular of from about 3 to about 10 μm.

27. The process as claimed in one of claims 17 to 26, characterized in that use is made of porous particles whose pores have an opening width of from about 0.1 to about 1000 nm, in particular of from about 3 to about 50 nm.

28. The process as claimed in one of claims 17 to 27, characterized in that use is made of particles which consist at least partially of silicate and/or latex.

29. The process as claimed in one of claims 17 to 28, characterized in that use is made of particles which possess a magnetic core.

30. A process for measuring the membrane permeation of substances, characterized in that
a) the substances are brought into contact, in one mixture, with porous particles as claimed in one of claims 1 to 16, and
b) after an incubation period, the quantity of the substances present within the particles is determined directly and/or indirectly.

31. The process as claimed in claim 30, characterized in that, after the incubation period, the particles are separated off from the mixture and the quantity of the substances within the particles and/or in the remaining mixture is determined.

32. The process as claimed in claim 30 or claim 31, characterized in that the substances are determined using chemical, radioactive or optical, in particular fluorimetric or luminometric, detection methods.

33. The use of porous particles as claimed in one of claims 1 to 16 for measuring membrane permeation.

34. A kit for measuring the membrane permeation of substances, comprising components for producing porous particles in accordance with a process as claimed in one of claims 17 to 29.

35. A kit for investigating membrane elements, in particular proteins, comprising components for producing porous particles in accordance with the process as claimed in one of claims 17 to 29.
